(19) United States
(12) Patent Application Publication
(10) Pub. No.: US 2023/0301557 A1
(43) Pub. Date: Sep. 28, 2023

(54) SYSTEMS, DEVICES AND METHODS FOR GLUCOSE SENSING AND ASSOCIATED METHODS

(71) Applicant: Tingo Medical Ltd., Tel Aviv (IL)

(72) Inventors: Lior STRAM, Tel Aviv (IL); Ofer YODFAT, Modi'in (IL)

(21) Appl. No.: 18/023,324

(22) PCT Filed: Aug. 26, 2021
(86) PCT No.: PCT/IL2021/051053

§ 371 Date: Feb. 24, 2023

Related U.S. Application Data
(60) Provisional application No. 63/070,735, filed on Aug. 26, 2020.
(60) Provisional application No. 63/072,050, filed on Aug. 28, 2020.

(51) Int. Cl. A61B 5/145; A61B 5/1486; A61B 5/1495

(52) U.S. Cl. CPC A61B 5/14532; A61B 5/14865; A61B 5/1495; A61B 2562/125; A61B 2562/166

(57) ABSTRACT

Embodiments of the present disclosure relate to systems, devices and methods for continuous glucose monitoring. In some embodiments, a continuous glucose monitoring (CGM) device is provided that includes a subcutaneous implantable electrochemical sensing probe (sensor). The sensor is planar and comprises counter and working electrodes that are deposited at both sides of the sensing probe. A biocompatible layer deposited at the entire sensor circumference minimizes inflammatory reaction. A reel to reel production process provides “per sensor” layers' deposition at predefined spots on a base sheet during displacement between production stations. Production processes are conducted initially at one side of the base sheet followed by production processes on the opposite side. Sensor by sensor working electrode surface areas and layers' thickness are measured and “per sensor” data (“dry” parameters) is stored according to base sheet identifiers that could be implemented by printed or etched markings or else by other means such as RFID. In addition, batch parameters are measured on a sample basis (in vitro “wet” parameters). A factory calibration algorithm analyzes “per lot” sample of sensors' performance (in vitro “wet” parameters in glucose solutions) as well as individual sensor measurement and combines the data in a calibration algorithm to calculate individual sensor calibration parameters and data is programmed back to the individual sensor.

### RELATED APPLICATIONS

This disclosure claims priority to and benefit of U.S. provisional application No. 63/072,050, filed Aug. 28, 2020, entitled, “Systems, Devices and Methods for Glucose Sensing and Associated Manufacturing Methods,” and U.S. provisional patent application No. 63/070,735, filed Aug. 26, 2020, entitled, “Continuous Glucose Sensor and Mounting Assembly.” Each of these disclosures in its entirety is incorporated herein by reference.

### FIELD OF DISCLOSURE

Embodiments of the present disclosure are, in general, directed to systems, devices and methods for continuous monitoring of glucose in a patient. More particularly, some embodiments of the present disclosure relate to a continuous glucose monitoring device having a planar subcutaneous glucose sensing probe. Even more particularly, some embodiments of the present disclosure relate to improved manufacturing process of a planar probe.

### BACKGROUND OF THE DISCLOSURE

Continuous glucose monitoring (CGM) is important to diabetes patients because it enables immediate changes to medication and/or lifestyle habits during times of rising or falling glucose levels. CGM devices detect glucose levels in the interstitial fluid but, in some devices, frequent blood glucose measurements (finger-sticks) are required for calibration.

CGM systems usually employ a subcutaneous implantable electrochemical sensing probe (sensor), which comprises a working and counter electrodes, and a skin adhered control unit that comprises the printed circuit board assembly (PCBA). An electrical current generated by enzymatic oxidation of glucose on the surface of the working electrode is correlated to interstitial glucose levels; a larger working electrode surface area increases sensor accuracy. The current is conducted to a skin adhered control unit and electrical circuit is closed by the sensor counter electrode. Both electrodes are usually covered with a biocompatible layer for reducing the foreign body inflammatory reaction.

CGM sensing probe is usually cylindrical or planar and both probes are inserted into the body with a removable introducer. A planar probe is preferable because insertion is less traumatic and production cost is low due to high yield of material usage and availability of high volume production methodologies and equipment. In most planar probes, the working and counter electrodes are deposited on one side of the probe limiting theirs surface area and sensor accuracy. Another limitation of planar probe is inflammatory reaction due to direct contact of the opposite side (no biocompatible layer) with the body.

Factory calibration of sensors takes away the calibration responsibility from the user and instead, places it in the hands of the sensor manufacturer. The sensor sensitivity is determined during the sensor manufacturing process, and that information is included with every sensor in the form of a sensor code. The main challenge for the manufacturer is to manufacture sensor lots with low sensor to sensor variability.

Thus, there is a need for a CGM device that is calibrated in the factory and eliminates finger-sticks and errors related to the execution of the calibration process. There is also a need for a manufacturing process that is robust with minimal lot variations. There is also a need for a planar probe that is miniature but provides maximal surface area for the electrodes. There is also a need for a planar probe that is biocompatible and causes minimal inflammatory reaction.

### SUMMARY

In some embodiments, a continuous glucose sensor is provided and comprises a base matrix sheet having at least one side, a counter electrode, and at least one working electrode. Such embodiments include one and/or another of (and in some embodiments, a plurality of, and in some embodiments, a majority of, substantially all of, or all of) the following features, functionality, steps, components, materials, parameters, and clarifications, yielding yet further embodiments:
the at least one side comprises a plurality of sides, and/or the base matrix sheet corresponds to a base matrix plate;
the plurality of sides include at least a first side;
the plurality of sides includes at least a first side and a second side;
the counter electrode is arranged on the first side;
the working electrode is arranged on a second side;
the second side is opposite to the first side;
the counter electrode and the working electrode are on a same side;
at least one of the counter electrode and working electrode are printed on the at least one side;
a reference electrode;
and
the at least one working electrode comprises two or more working electrodes.

In some embodiments, a method for production of a continuous glucose monitoring device (CGM device) sensor is provided, and comprises providing an extended length of base matrix sheet, or individual pieces of base matrix sheet and pursuing a first process comprising displacing the extended length of base sheet through a plurality of production stations, where at each station, the base sheet undergoes at least one production process, the at least one production process at least including deposition of an enzyme layer, and screen printing of dielectric layer, on at least one side of the base sheet, and at least one of folding and cutting the extended length into individual sensors; or a second process comprising processing the individual pieces of the base sheet, one at a time, to produce a single sensor, or a plurality of sensors, where the plurality of sensors is selected from the group consisting of: 2 or more sensors, 2-5, sensors, and 2-10 sensors (in some embodiments, between 2-100 sensors, between 2-1000 sensors, between 2-10,000 sensors, and all ranges therebetween), where processing of the individual pieces includes at least deposition of an enzyme layer, and screen printing of dielectric layer, on at least one side of the piece of base sheet.

Such embodiments include one and/or another of (and in some embodiments, a plurality of, and in some embodiments, a majority of, substantially all of, or all of) the following features, functionality, steps, components, materials, parameters, and clarifications, yielding yet further embodiments:
the sensor corresponds to the sensor of embodiments disclosed herein;
the at least one production process is performed on a first side and a second side of the base sheet;
effecting at least one and preferably a plurality of fiducial markings for use in at least one of: alignment of specific deposition areas/points/spots, and markings for coding and/or tacking of the CGM device, on one or two sides of the base sheet, where optionally, in place of markings for coding and/or tracking of the CGM device, an RFID tag can be coupled to the base sheet;
the at least one production process is performed on a first side, then a second side;
the first side or bottom side is configured as a counter electrode side for including the counter electrode, and the second side to upper side is configured as a working electrode side for including the working electrode, the second side being opposite the first side;
covering at least one portion of the base sheet on at least one and preferably both of the first and second sides;
the at least one portion of the base sheet comprises a strip of the base strip bordered by an edge thereof;
the covering comprises a metal;
the metal comprises at least one of gold and platinum;
the metal is a sputtered metal;
providing a liner material configured to protect at least one edge area of the base sheet;
for the counter electrode side, at one and/or another of the production stations, depositing, at predetermined locations, at least one of and preferably a plurality of, and more preferably all of an Ag/AgCl layer, a dielectric layer and a biocompatible layer;
for the working electrode side, the process includes at least one of, and preferably a plurality of, and more preferably all of deposition of an enzyme layer, a dielectric layer and a biocompatible layer, an interface conductive layer, a glucose limiting layer, and an anti-interference layer;
the biocompatible layer is deposited on top of one or more other layers, and/or at both sides through one or more bilateral slots provided in the base sheet, where optionally, the deposition of the biocompatible layer from the top and sides is configured to cover a circumference of the sensor circumference;
cutting the base sheet into one or more planar portions/configurations;
the base sheet further includes electrical contacts in electrical communication with each electrode, and the method further comprises at least one of, and preferably a plurality of, and more preferably, all of folding the base sheet at approximately 90°, coupling the base sheet with an introducer, and coupling the electrical contacts with a printed circuit board assembly of a skin adhered control unit;
the base sheet includes a plurality of pre-specified areas or points/spots for the deposition of materials one or two sides thereof;
during the second process, each piece of base sheet is stationary, where stationary can comprise processing each piece of base matrix at a single location;
for the second process, material deposition is conducted by displacement of a material injector to a plurality of areas/points/spots over the piece of base sheet;
calibrating each sensor;
curing during and/or after deposition of one or more layers on one or both sides of the base sheet;
the base sheet thickness is between 25-75 um (and any range therebetween);
the/a conductive layer includes a thickness of between 50-200 nm (and any range therebetween);
covering one or both sides of the base sheet with a protective liner;
and
depositing at least one additional layer selected from the group consisting of: enzymes, mediators, cross linkers, adhesives, and any polymer that can be used for controlling diffusion of glucose and oxygen and/or for controlling the diffusion of various interfering compounds, including, optionally, acetaminophen, and ascorbic acid.

In some embodiments, a CGM device sensor calibration method is provided and comprises confirming sensor-by-sensor parameters to produce calibration data, where such confirmation is via lot measurements of a sampling of select number of produced sensors (e.g., between 1 and 100), and the parameters are selected from the group consisting of: a surface area of the working electrode, a thickness of one or more specific layers, the thickness of all layers.

Such embodiments include one and/or another (and in some embodiments, a plurality of, and in some embodiments, all of the following features, functionality, steps, components, materials, parameters, and clarifications, yielding yet further embodiments:
the calibration data for each sensor is stored and associated with a specific sensor;
conducting in-vitro measurements of one or more sensors to record sensor performance to produce in-vitro calibration data;
providing at least one of, and preferably both of, dry calibration data, and in-vitro calibration to a calibration algorithm, and/or producing sensor calibration data configured for use in electronic circuitry of a continuous glucose monitor device and/or system;
producing single sensor production data;
producing single sensor production data comprise using an imaging device;
the imaging device images single sensors, and the imaging device may comprise a microscope/imager;
via at least one of an optical profiler, spectral reflectance monitor, and the like, producing single sensor production calibration data;
and
prior to production, measuring one or more properties of one or more materials used in production of one or more sensors.

Some of the embodiments of the present disclosure relate to methods and devices for continuous glucose monitoring (CGM) using a subcutaneous planar electrochemical sensing probe (sensor). Some embodiments also relate to the production process of the CGM device, and some embodiments also relate to factory calibration processes. In some embodiments, the CGM device includes a planar matrix base sheet that comprises electrodes, conductors, dielectrics, and electrical contacts deposited on both sides of a planar sheet. The matrix base sheet can be folded 90°, such that a portion of it is configured to be inserted in a body (sensor) and another portion, which may be configured to be included in a skin, adhered control unit of the CGM device.

The control unit, according to some embodiments, includes a printed circuit board assembly (PCBA) that is electrically coupled with matrix base plate contacts. In some embodiments, the planar sensor includes a counter electrode that is deposited on one side of the sensor, and at least one working electrode that is deposited on the opposite side of the sensor. The counter electrode includes, in some embodiments, an Ag/AgCl layer, and the working electrode, in some embodiments, includes at least one enzyme layer and may also include at least one of a glucose limiting layer and an anti-interference layer. In some embodiments, both the counter electrode and the working electrode, and in some embodiments as well as edges and electrode a biocompatible layer that minimizes reactive inflammatory reaction protects sides.

In some embodiments, a production process for the matrix base sheet comprises a “reel to reel” process. Specifically, a long strip of a base sheet, which may comprise polyimide, is displaced through production stations, where, at each station, the base sheet undergoes an at least one production process, such as, deposition of an enzyme layer, screen printing of dielectric layer, etc. The polyimide (for example) base sheet, in some embodiments, includes fiducials for alignment of specific deposition areas, which may also be referred to as spots or points, and such terms being used interchangeably throughout, and markings for a sensor by sensor coding; alternatively individual sensors can be identified by other means such as RFID, for example. The production process can be, in some embodiments, conducted initially at one side of the base sheet (e.g., a counter electrode side or bottom side), and then on another side, which in some embodiments, is a side opposite to the side counter electrode side (e.g., a working electrode side or upper side). The base sheet strip, in some embodiments, is initially covered on both sides (bottom and lower sides) by a conductive sputtered metal (e.g., gold, platinum, etc.) and, in some embodiments, protected with bottom side and upper side liners.

The production process at the counter electrode side includes, in some embodiments, for example, deposition at specified locations of at least an Ag/AgCl layer, a dielectric layer and a biocompatible layer. The production process at the working electrode side (after flipping of base strip sheet) includes, in some embodiments, for example, deposition of at least an enzyme layer, a dielectric layer and a biocompatible layer, but may additionally include, in some embodiments, at least one of (and in some embodiments, a plurality of, and in some embodiments, all of) an interface conductive layer (such as carbon), a glucose limiting layer, and an anti-interference layer. The biocompatible layer, in some embodiments, is deposited on top of one or more other layers, and at both sides of the sensor electrodes through bilateral slots in the base sheet. Following the deposition of layers' deposition on both sides, in some embodiments, the matrix base sheet is cut (e.g., by laser cutting) to a planar configuration. The matrix base sheet, in some embodiments, is then folded at 90°, the sensor is coupled with the introducer, and electrical contacts are coupled with the PCBA within a/the skin adhered control unit. In some embodiments, the production process of the matrix sheet employs a rectangular base sheet having a length and a width, where each sheet includes hundreds or thousands pre-specified areas/spots for materials' deposition. Unlike a reel to reel process, the base sheet is stationary and “per sensor” material deposition is conducted by displacement of the material injector—for example, displacement of a liquid deposition syringe for deposition of enzyme at multiple working electrode areas/spots.

Manufacture/factory calibration of sensors of the present disclosure, and is some embodiments, is provided by, in process, sensor-by-sensor dimension measurements (e.g., dry measurements) by, for example, a camera, and sampling of sensor lot measurements in glucose solutions (e.g., dry measurements of sensor performance). Sensor-by-sensor dry parameters include, at least one of, the surface area of the working electrode and the thickness of one or more specific layers. In some embodiments, data for each sensor is stored according to a specific sensor identified. Furthermore, in some embodiments, batch parameters are measured by extracting samples from a given batch and conducting in vitro (“wet”) measurements to record sensor performance. Thus, in some embodiments, the combination of sensor specific measurements together with batch measurements is provided to at least one calibration algorithm in order to produce at least one, and preferably a plurality of, sensor-by-sensor calibration parameters. The resulting sensor calibration parameter(s) can then be programed into/be a part of, electronic circuitry of individual sensor devices after assembly is completed.

These and other embodiments, and objects and/or advantages thereof, will become even more apparent with reference to the attached figures, and detailed description which follows.

### BRIEF DESCRIPTION OF DRAWINGS

FIGS. 1a-c show cross-sectional (1a) and side views (1b and 1c) of a sensing probe, according to some embodiments of the present disclosure.

FIG. 2a shows a scheme of the sensor matrix base plate before folding into its final spatial configuration, according to some embodiments of the present disclosure.

FIG. 2b shows the matrix base plate in a final spatial configuration after folding, according to some embodiments of the present disclosure.

FIG. 3 shows a reel-to-reel production process, according to some embodiments of the present disclosure.

FIG. 4 shows a cross-sectional view of a base sheet that comprises upper and bottom sputtered metal layers, each layer is covered with a liner, according to some embodiments of the present disclosure.

FIG. 5 shows a base sheet (counter electrode side) after deposition of bottom dielectric at defined locations, according to some embodiments of the present disclosure.

FIG. 6 shows a base sheet (counter electrode side) after deposition of Ag/AgCl at defined locations, according to some embodiments of the present disclosure.

FIG. 7 shows a base sheet (counter electrode side) after selective laser etching creating a matrix base plate outlines, fiducials, and individual sensor markings and after deposition of biocompatibility layer and curing, according to some embodiments of the present disclosure.

FIG. 8a-b show a base sheet (counter electrode side) after deposition of pressure sensitive adhesive (PSA, 17) (8a) and adhering a second bottom side liner 18 (8b), according to some embodiments of the present disclosure.

FIG. 9 shows removal of upper liner from a base sheet, according to some embodiments of the present disclosure.

FIGS. 10a-b show a base sheet (working electrode side) after deposition of carbon electrodes 40 (FIG. 10a) and upper side dielectrics 22 (FIG. 10b), and laser etching of the matrix base plate outline, according to some embodiments of the present disclosure.

FIG. 11 shows the image capture of a working electrode surface area, according to some embodiments of the present disclosure.

FIG. 12 shows the various layers that are deposited on a bottom (counter electrode side) and upper (working electrode side) of a base sheet, according to some embodiments of the present disclosure. The height (thickness) of each layer is measured.

FIG. 13 shows the base sheet (working electrode side) after deposition of a biocompatible layer, according to some embodiments of the present disclosure. The biocompatible polymer is deposited on top and on both sides of each working electrode.

FIG. 14 shows a base sheet after cutting and releasing of individual matrix base plates, according to some embodiments of the present disclosure.

FIGS. 15a-e show a folding process of matrix base plate and assembly with introducer, according to some embodiments of the present disclosure. Accordingly, the planar matrix base plate can be coupled with the introducer (FIGS. 15a-b), folded in 90° relative to the sensing probe (FIG. 15c) and, may then be folded 180° at one side (FIGS. 15d-e).

FIG. 16 shows a flow chart of a calibration process, according to some embodiments of the present disclosure.

### DETAILED DESCRIPTION

Terminology used, and associated reference numbers, corresponding to at least some of the embodiments.
10 Base sheet (kapton)
11 Bottom side sputtered metal layer
12 Upper side sputtered metal layer
13 First bottom side liner
14 Upper side liner
15 Fiducials
16 Markings
17 Pressure sensitive adhesive
18 Second bottom side liner
20 Sensor matrix base plate
21 Bottom dielectric (counter electrode side)
22 Upper side dielectric (working electrode side)
23 Matrix base plate fold
30 Counter electrode
31 Ag/AgCl layer
40 Working electrode (carbon)
41 Modified SPC layer
42 Enzyme layer
43 Glucose limiting layer
50 Biocompatible layer
51 Biocompatible layer bottom side (counter electrode side)
52 Biocompatible layer upper side (working electrode side)
60 Sensor probe
61 Sensor probe tip
70 Skin adhered control unit
71 PCBA
72 Conductive spring
80 Skin
90 Introducer
100 Camera

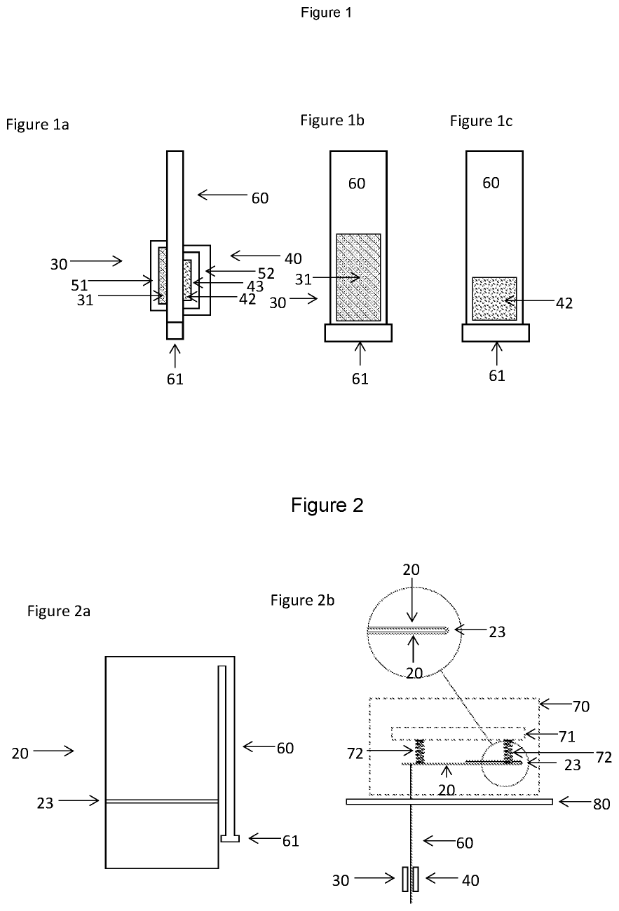

FIGS. 1a-c show cross-sectional (1a) and side views (1b and 1c) of a sensing probe (hereinafter “sensor”) according to some embodiments. The sensor 60, in some embodiments, is planar and can include two sides and a sensor probe tip 61, where each side may include at least one electrode. In some embodiments, the sensor 60 includes 2 electrodes, a counter electrode 30 which can be deposited on one side (a first, or bottom side) and a working electrode 40 which can be deposited on an opposite side (second, or upper side). In some embodiments, the counter electrode 30 includes an Ag/AgCl layer 31 and a biocompatible layer 51. The working electrode, in some embodiments, can include at least one enzyme layer 42, and in some embodiments, also includes a glucose limiting layer 43, and a biocompatible layer 52. In some embodiments, two or more working electrodes 40 can be deposited on one side and the counter electrode 30, (which, in some embodiments, is larger) can be deposited on an opposite side.

In some embodiments, an additional conductive layer can be deposited on the working electrode, and comprise a sputtered metal layer, such as, for example, carbon, and/or modified carbon (e.g., carbon modified with Prussian blue, carbon modified with ruthenium purple, etc.). In some embodiments, an additional layer can be added, which may be an anti-interference layer, either above or below the enzyme layer (42).

FIG. 2a shows a scheme of the sensor matrix base plate 20 before folding into its final spatial configuration, according to some embodiments. The matrix base plate 20 can comprise at least one of (and in some embodiments, a plurality of, and in some embodiments, all of) sensing probe 60, sensor tip 61, and matrix base plate fold 23. The matrix base plate 20 can include (in some embodiments, on both sides, which is not shown) sensor electrodes (counter and working), conductors, and dielectrics.

FIG. 2b shows the matrix base plate 20 in a final (according to some embodiments) spatial configuration after folding. The sensor 60 is preferably folded 90° relative to base plate 20, and preferably 180° at one side at the matrix plate fold 23 (magnified view). In a final spatial configuration, according to some embodiments, a portion of the matrix base plate 20 is configured to be included in skin adhered control unit 70, and another portion, sensing probe 60, is configured to be inserted through the skin 80 into the subcutaneous tissue. The sensing probe 60, in some embodiments, includes counter electrode 30 on one side, and working electrode 40 on a second/other side, which in some embodiments, is a side oppose to the side having the counter electrode. In some embodiments, electrical current that is generated on working electrode 40 at one side of sensor 60 can be conducted via one side (e.g., bottom side) of matrix plate 20, matrix plate fold 23, folded matrix plate 20 (facing up), and one conducting spring 72, to the PCBA 71. The electrical circuit, in some embodiments, is closed via a second conducting spring 72, opposite side (e.g., upper side) of matrix base plate 20, and counter electrode 30 that is deposited on the opposite side of the sensor 60. In some embodiments, the position of counter electrode 30 and working electrode 40 is reversed.

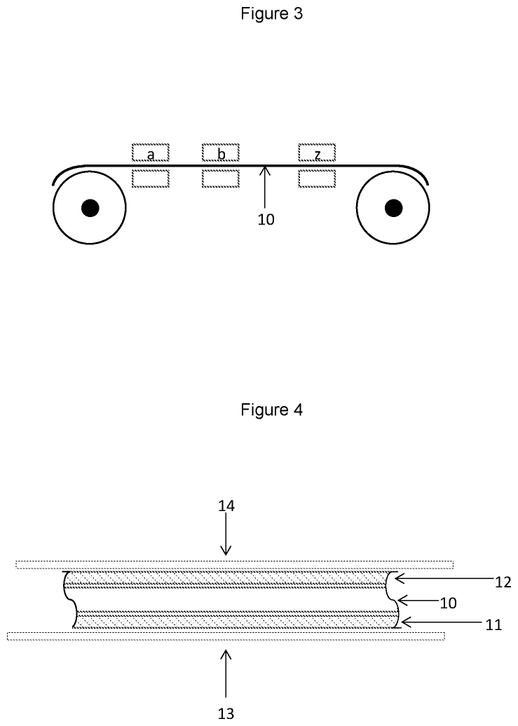

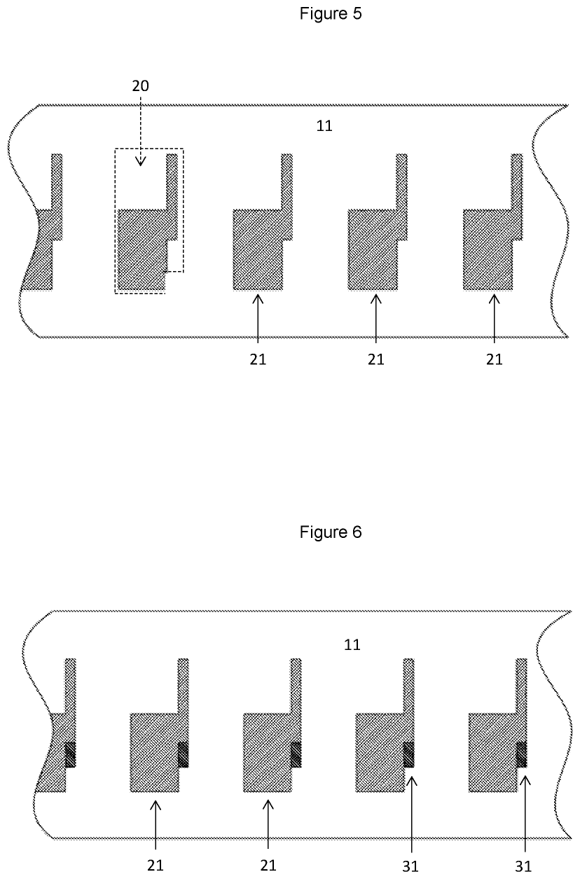

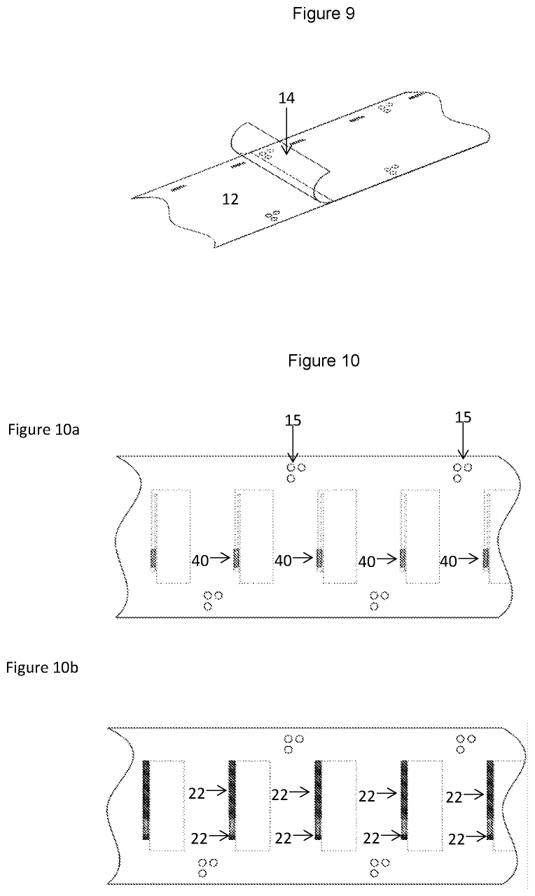

FIGS. 3-15 show aspects of a production process (step-by-step) of a planar matrix base plate and sensing probe (sensor) including layer deposition, curing, measurements of sensor by sensor parameters, and cutting the product (planar matrix base sheet and sensing probe) from the base sheet. In some embodiments, selective deposition (at specific spots) of layers at predetermine surface area can be done by any known in the art technique such as screen printing, photolithography, and the like. FIGS. 5-8 show production processes, according to some embodiments, of the counter electrodes side (e.g., bottom side) and FIGS. 9-15 show production processes, according to some embodiments, of the working electrodes side (e.g., upper side). The process outlined in each figure can also represent an embodiment of the disclosure.

FIG. 3 shows a production process—a reel-to-reel process, according to some embodiments. Here, base sheet 10 is configured as a long strip that is spanned between two reels. Predefined spots on base sheet 10 undergo sequential production processes at production stations a, b . . . z during displacement of the base sheet (in FIG. 3, from right to left). At each production station (a, b . . . z), one spot of the base sheet undergoes specific production process (for example deposition of enzyme layer on the working electrode). In some embodiments (not shown), the base sheet can be configured as a planar rectangular sheet having a length and a width, and includes hundreds or thousands of predefined spots. At each production station (a, b . . . z), the specific production process can be repeated for all spots on the sheet (for example deposition of enzyme layer is repeated for all hundreds or thousands spots).

FIG. 4 shows a longitudinal cross-sectional view of the base sheet 10, according to some embodiments. The base sheet 10 can be a polyimide (kapton), or any other thin polymer film that are currently used in the electronics industry. Accordingly, the base sheet 10 is covered with a conductor on both sides, e.g., sputtered metal such as gold or platinum. The base sheet thickness can be between 25-75 um (and any range therebetween), and each sputtered conductive layer can include a thickness can be between 50-200 nm (and any range therebetween). At least one of, and in some embodiments, the sputtered metal layers, at the bottom side 11 and upper side 12, can be covered with a protective liner—bottom liner 13 and upper liner 14, respectively.

FIG. 5 shows a base sheet (counter electrode side or bottom side), according to some embodiments, that is covered with a sputtered metal layer 11 after removal of bottom liner and deposition of bottom dielectric 21 at defined spots. A final shape of the matrix base plat 20 is shown in dashed line.

FIG. 6 shows the bottom side of the base sheet that is covered with a sputtered metal layer 11 after deposition of dielectric 21 and deposition of Ag/AgCl layer 31 at defined spots, according to some embodiments. The Ag/AgCl layer, in some embodiments, is the basis of the counter electrode.

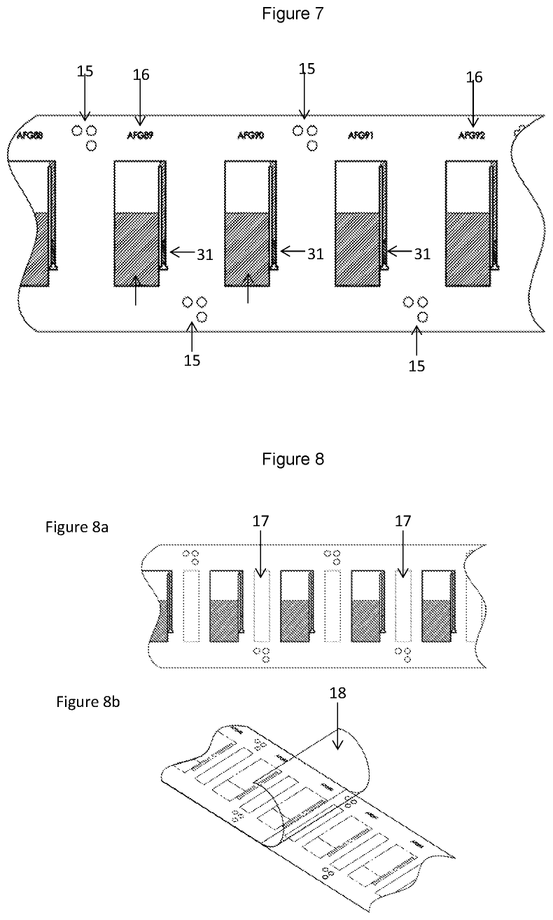

FIG. 7 shows the base sheet (counter electrode side) after selective laser etching creating the matrix base plate outlines, fiducials 15, and individual sensor identifier 16 (e.g., markings), according to some embodiments. Following deposition of the dielectric and Ag/AgCl layer 31, a laser etching process individuates the sensors and adds markings such as fiducials and unit ID. In case RFID technology is used to identify individual sensors, an RFID chip is coupled to the substrate using a same area as illustrated by individual sensor markings. Following the laser etching process, in some embodiments, a biocompatible layer is deposited and cured. Curing of the biocompatible layer can be, in some embodiments, a final production process of the counter electrode.

FIG. 8a-b show a base sheet (counter electrode side) after deposition of pressure sensitive adhesive (PSA, 17) (8a) and adhering a second bottom side liner 18 (8b), according to some embodiments. The second bottom side liner provides rigidity to the base sheet during the production processes of the working electrode on the opposite side of the base sheet. At the end of this process the base sheet is flipped to the working electrode side (upper side).

FIG. 9 shows removal of upper side liner 14 from the upper side sputtered metal layer 12, according to some embodiments.

FIGS. 10a-b show a base sheet (working electrode side) after deposition of carbon/working electrodes 40 (10a) and upper side dielectrics 22 (10b), and laser etching of the matrix base plate outline, according to some embodiments. The sheet can be aligned by the fiducials 15 that define spots for deposition of carbon electrodes 40 and/or upper side dielectrics 22. Following deposition of the carbon electrodes 40, an exact surface area of each electrode 40 can be measured and associated data per sensor (according to the individual sensor identifier), can be stored.

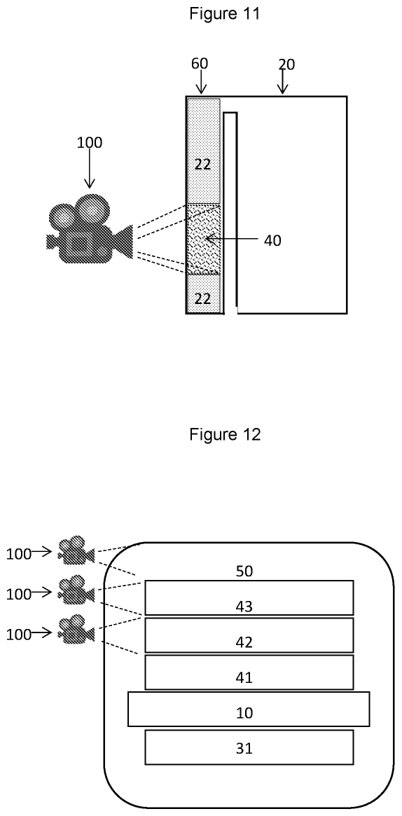

FIG. 11 shows image capture of the surface area of working electrodes 40, according to some embodiments. Working electrode 40 can be deposited on planar sensing probe 60, which can also include dielectrics 22. Sensing probe 60 is, in some embodiments, part of planar matrix base plate 20. A camera 100 can be included to capture working electrode 40 surface area and data associated therewith (per sensor identifier), where such data can be stored.

FIG. 12 shows various layers that can be deposited on one side, e.g., a bottom (counter electrode side), and another side, e.g., upper (working electrode side) of base sheet 10, according to some embodiments. Counter electrode side (e.g., bottom side), can include Ag/AgCl layer 31, and biocompatible layer 50, and, working electrode side can include modified SPC (stone plastic composite) layer 41 (e.g., modified SPC layer), enzyme layer 42, glucose limiting layer 43, and biocompatible layer 50. The layers shown in FIG. 12 are just one example of the composition of layers used for continuous glucose sensing. Other examples may include, for example, any combination of layers including enzymes, mediators, cross linkers, adhesives, and any polymer that can be used for controlling diffusion of glucose and oxygen (as well as controlling the diffusion of various interfering compounds, including, for example, acetaminophen, ascorbic acid, etc.). Deposition of layers can be done by any process known in the art including, for example, liquid deposition using syringe, inkjet printing, screen printing, etc. Following the deposition of layers, a height (i.e., thickness) of the layers can be captured, and the sensor-by-sensor data can be stored (per sensor identifier). The layers' height (thickness) measurement can be done with optical profiler, spectral reflectance monitor, and/or the like.

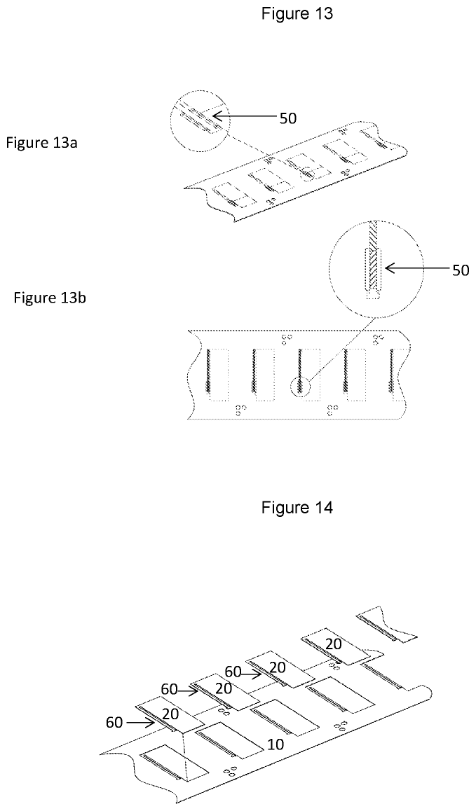

FIG. 13 shows the base sheet (working electrode side) after deposition of the biocompatible layer 50, according to some embodiments. The biocompatible layer 50 can be deposited on top and on both sides of each working electrode. Deposition of the biocompatible layer 50 on both sides of the working electrode can be provided by slots in the base sheet; the biocompatible polymer can deposited on top of the working electrode and can also be deposited in both slots, and can be coupled with the biocompatible layer that was deposited on the counter electrode on the opposite side of the base sheet.

FIG. 14 shows base sheet 10 after cutting (for example, laser cutting or die cutting) and releasing of the individual planar matrix base plates 20 and its sensing probe 60, according to some embodiments.

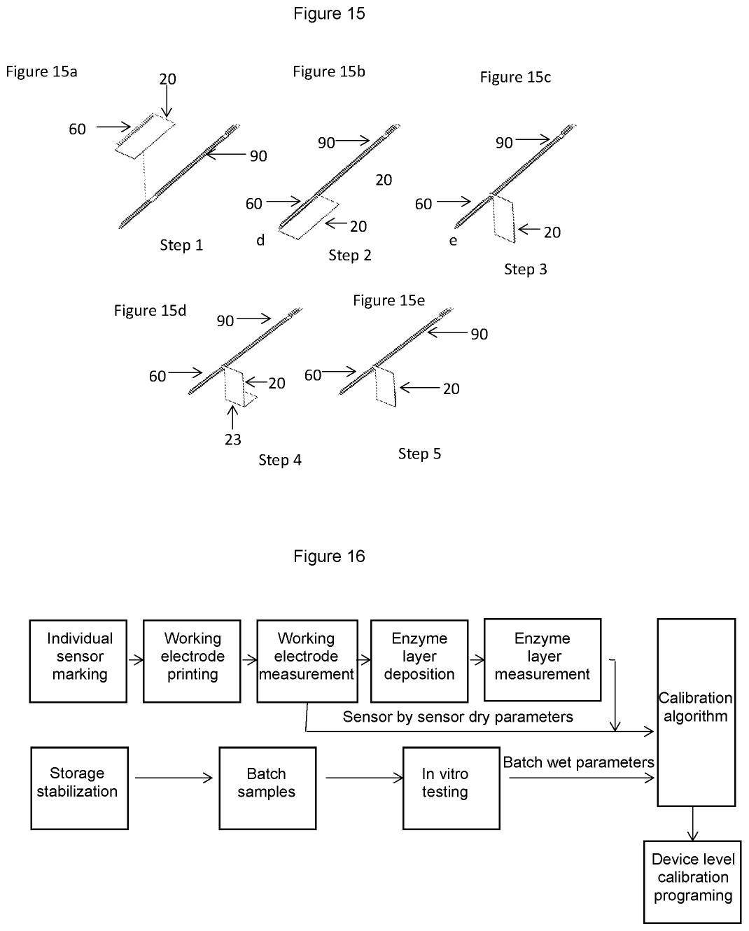

FIGS. 15a-e show a folding process of matrix base plate 20 and assembly with introducer 90, according to some embodiments. Accordingly, the planar matrix base plate 20 can be coupled with the introducer (FIGS. 15a-b), folded in 90° relative to the sensing probe 60 (FIG. 15c) and, may then be folded 180° at one side (FIGS. 15d-e), resulting in matrix base plate 20 within the skin adhered controlling unit of the CGM, which is shown in FIG. 2.

FIG. 16 shows a flow chart of the factory calibration process, according to some embodiments. The process includes measurement of “per single sensor” one or more (and preferably a plurality) of parameters that are captured during the production process for each sensor (e.g., sensor-by-sensor “dry” parameters) and “per batch sampled sensors” measurement of parameters that are captured by in-vitro testing of sensors in solutions with various glucose concentrations (batch “wet” parameters), according to some embodiments. In-vitro testing can be conducted after batch production is completed and sensors being stored until stabilization. “Dry per sensor” parameters can include “in process” measurements of surface area of a single sensor working electrode and thickness of at least one layer of the sensor working electrode. Sensors can be identified by either “per sensor” specific identifier that are printed on the base sheet, and/or using electronic means as shown in FIG. 7. Data stored that was gathered from both the “dry” and “wet” measured parameters is analyzed by the calibration algorithm and coded in each specific CGM device.

While various inventive embodiments have been described and illustrated herein, those of ordinary skill in the art will readily envision a variety of other means and/or structures for performing the function, and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, steps, and configurations described herein are meant to be merely an example and that the actual parameters, dimensions, materials, steps, and configurations will depend upon the specific application or applications for which the inventive teachings is/are used. Those skilled in the art will recognize, or be able to ascertain using no more than routine experimentation, many equivalents to the specific inventive embodiments described herein. It is therefore to be understood that the foregoing embodiments are presented by way of example only and that, within the scope of claims supported by the subject disclosure and equivalents thereto, and inventive embodiments may be practiced otherwise than as specifically described and claimed. Inventive embodiments of the present disclosure are directed to each individual feature, device, system, article, material, kit, step, function/functionality, and method described herein. In addition, any combination of two or more such features, devices, systems, articles, materials, kits, steps, functions/functionality, and methods, if such features, systems, articles, materials, kits, steps, functions/functionality, and methods are not mutually inconsistent, is included within the inventive scope of the present disclosure, and considered embodiments.

Embodiments disclosed herein may also be combined with one or more features, as well as complete systems, devices, and/or methods, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, as noted, various inventive concepts may be embodied as one or more methods, of which one or more examples have been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is currently claimed:

1. A method for production of a continuous glucose monitoring device (CGM device) sensor, comprising providing an extended length of base matrix sheet, or individual pieces of base matrix sheet, and pursuing a:
a first process comprising displacing the extended length of base sheet through a plurality of production stations, wherein:
at each station, the base sheet undergoes at least one production process,
the at least one production process at least including:
deposition of an enzyme layer,
screen printing of dielectric layer on at least one side of the base sheet,
and
at least one of folding and cutting the extended length into individual sensors;
or
a second process comprising processing the individual pieces of the base sheet, one at a time, to produce a single or a plurality of sensors,
wherein:
the plurality of sensors is selected from the group consisting of: 2 or more sensors, 2-5, sensors, 2-10 sensors, 2-100 sensors, and 2-1000 sensors, and
processing of the individual pieces includes at least deposition of an enzyme layer, and screen printing of dielectric layer, on at least one side of the piece of base sheet.

2. The method of claim 1, wherein the sensor corresponds to the sensor of any of claims 37-47.

3. The method of claim 1, wherein the at least one production process is performed on a first side and a second side of the base sheet.

4. The method of claim 1, further comprising effecting at least one and preferably a plurality of fiducial markings for use in at least one of alignment of specific deposition areas/points/spots and markings for coding and/or tacking of the CGM device, on one or two sides of the base sheet, wherein optionally, in place of markings for coding and/or tracking of the CGM device, an RFID tag can be coupled to the base sheet.

5. The method of any of claims 1-4, wherein the at least one production process is performed on a first side, then a second side.

6. The method of any of claims 1-5, wherein the first side or bottom side is configured as a counter electrode side for including the counter electrode, and the second side to upper side is configured as a working electrode side for including the working electrode, the second side being opposite the first side.

7. The method of any of claims 1-6, further comprising covering at least one portion of the base sheet on at least one and preferably both of the first and second sides.

8. The method of claim 18, wherein the at least one portion of the base sheet comprises a strip of the base strip bordered by an edge thereof.

9. The method of claim 7 or 8, wherein the covering comprises a metal.

10. The method of claim 9, wherein the metal comprises at least one of gold and platinum.

11. The method of claim 9 or 10, wherein the metal is a sputtered metal.

12. The method of any of claims 1-11, further comprising providing a liner material configured to protect at least one edge area of the base sheet.

13. The method of any of claims 1-12, wherein, for the counter electrode side, the method includes, at one and/or another of the production stations, depositing, at predetermined locations, at least one of and preferably a plurality of, and more preferably all of an Ag/AgCl layer, a dielectric layer and a biocompatible layer.

14. The method of any of claims 1-13, wherein, for the working electrode side, the process includes at least one of, and preferably a plurality of, and more preferably all of deposition of an enzyme layer, a dielectric layer and a biocompatible layer, an interface conductive layer, a glucose limiting layer, and an anti-interference layer.

15. The method of claim 14, wherein the biocompatible layer is deposited on top of one or more other layers, and/or at both sides through one or more bilateral slots provided in the base sheet, wherein optionally, the deposition of the biocompatible layer from the top and sides is configured to cover a circumference of the sensor circumference.

16. The method of any of claims 1-15, further comprising cutting the base sheet into one or more planar portions/configurations.

17. The method of any of claims 1-16, wherein the base sheet further includes electrical contacts in electrical communication with each electrode, and wherein the method further comprises at least one of, and preferably a plurality of, and more preferably, all of folding the base sheet at approximately 90°, coupling the base sheet with an introducer, and coupling the electrical contacts with a printed circuit board assembly of a skin adhered control unit.

18. The method of any of claims 1-17, wherein the base sheet includes a plurality of pre-specified areas or points/spots for the deposition of materials one or two sides thereof.

19. The method of claim 1, wherein during the second process, each piece of base sheet is stationary.

20. The method of claim 19, wherein stationary comprises processing each piece of base matrix at a single location.

21. The method of claim 19, wherein for the second process, material deposition is conducted by displacement of a material injector to a plurality of areas/points/spots over the piece of base sheet.

22. The method of claim 1, further comprising calibrating each sensor.

23. The method of claims 1-22, further comprising curing during and/or after deposition of one or more layers on one or both sides of the base sheet.

24. The method of any of claims 1-23, wherein the base sheet thickness is between 25-75 um.

25. The method of any of claims 1-24, wherein the/a conductive layer includes a thickness of between 50-200 nm.

26. The method of any of claims 1-25, further comprising covering one or both sides of the base sheet with a protective liner.

27. The method of any of claims 1-26, further comprising depositing at least one additional layer selected from the group consisting of: enzymes, mediators, cross linkers, adhesives, and any polymer that can be used for controlling diffusion of glucose and oxygen and/or for controlling the diffusion of various interfering compounds, including, optionally, acetaminophen, and ascorbic acid.

28. A CGM device sensor calibration method comprising:
confirming sensor-by-sensor parameters to produce calibration data, wherein:
such confirmation is via lot measurements of a sampling of select number of produced sensors, and
the parameters are selected from the group consisting of: a surface area of the working electrode, a thickness of one or more specific layers, the thickness of all layers.

29. The calibration method of claim 28, wherein the calibration data for each sensor is stored and associated with a specific sensor.

30. The calibration method of claim 28 or 29, further comprising conducting in-vitro measurements of one or more sensors to record sensor performance to produce in-vitro calibration data.

31. The calibration method of any of claims 28-30, further comprising providing at least one of, and preferably both of, dry calibration data, and in-vitro calibration to a calibration algorithm, and producing sensor calibration data configured for use in electronic circuitry of a continuous glucose monitor device and/or system.

32. The calibration method of any of claims 28-31, further comprising producing single sensor production data.

33. The calibration method of claim 32, wherein producing single sensor production data comprise using an imaging device.

34. The calibration method of claim 32 or 33, wherein the imaging device images single sensors, and the imaging device may comprise a microscope/imager.

35. The calibration method of any of claims 28-34, further comprising, via at least one of an optical profiler, spectral reflectance monitor, and the like, to produce single sensor production calibration data.

36. The calibration method of any of claims 28-34, further comprising, prior to production, measuring properties of material used in production.

37. A continuous glucose sensor comprising:
a base matrix sheet having at least one side;
a counter electrode; and
at least one working electrode.

38. The sensor of claim 37, wherein the at least one side comprises a plurality of sides, and/or the base matrix sheet corresponds to a base matrix plate.

39. The sensor of claim 38, wherein the plurality of sides include at least a first side.

40. The sensor of claim 38 or 39, wherein the plurality of sides includes at least a first side and a second side.

41. The sensor of claim 39 or 40, wherein the counter electrode is arranged on the first side.

42. The sensor of any of claims 39-41, wherein the working electrode is arranged on a second side.

43. The sensor of any of claims 40-42, wherein the second side is opposite to the first side.

44. The sensor of claim 37 or 38, wherein the counter electrode and the working electrode are on a same side.

45. The sensor of any of claims 37-44, wherein at least one of the counter electrode and working electrode are printed on the at least one side.

46. The sensor of any of claims 37-45, further comprising a reference electrode.

47. The sensor according to any of claims 36-46, wherein the at least one working electrode comprises two or more working electrodes.

48. A system, device, or method according to any of the disclosed embodiments.
